(19) United States
(12) Patent Application Publication
(10) Pub. No.: US 2011/0213380 A1
(43) Pub. Date: Sep. 1, 2011

(54) INTRAOCULAR LENS INJECTION DEVICE IN WHICH INTRAOCULAR LENS IS PRELOADED

(75) Inventor: Myoung Soo Han, Cheongju-si (KR)

(73) Assignee: RET CO., LTD., Chungcheongbuk-do (KR)

(21) Appl. No.: 12/665,221

(22) PCT Filed: Sep. 18, 2009
(86) PCT No.: PCT/KR2009/005341

§ 371 Date: Dec. 17, 2009

(30) Foreign Application Priority Data
Sep. 19, 2008 (KR) 10-2008-0092070

(51) Int. Cl. A61F 9/007

(52) U.S. Cl. 606/107

(57) ABSTRACT

Disclosed herein is a disposable intraocular lens injection device, in which a receiving portion which is sealed after an intraocular lens is placed in an unfolded state therein, a cylinder including an injection guide disposed in front of the receiving portion and allowing the unfolded intraocular lens to be provided to a patient in a rolled state, and a plunger slidably disposed in the cylinder and injecting the intraocular lens in the receiving portion through the injection guide are packaged together with the injector device such that, since the intraocular lens is preloaded in the injection device, the difficulty for an operator to load the intraocular lens in the injection device during operation is overcome, thus making rapid progress while preventing bacteria infection during the operation.

### TECHNICAL FIELD

The present invention relates to an intraocular lens injection device used during cataract surgery and, more particularly, to a disposable intraocular lens injection device in which an intraocular lens is preloaded.

### BACKGROUND ART

Cataract is a disease caused by opacity of the crystalline lens of the eye and may cause blindness in a severe case. The cataract is classified into a congenital cataract present in utero or at birth, a senile cataract occurring in the aged, a generalized cataract caused by various diseases, and the like. Among them, the senile cataract is most frequently observed in the aged.

Methods for treatment of cataract include pharmacotherapy and surgery. Since there is no guarantee that the pharmacotherapy can completely cure the cataract, the cataract surgery is mainly used.

Cataract surgery methods include intracapsular extraction, in which the entire crystalline lens and its enclosing capsule are removed, and extracapsular extraction, the posterior capsule of the crystalline lens having a thickness of about 0.01 mm, which is adjacent to the hyaloid, is left in place and the remaining crystalline lens is removed.

The intraocular lens is an artificial lens used to replace the natural crystalline lens of the eye when the natural lens has cataracts or is otherwise diseased. The intraocular lens is also sometimes implanted into an eye to correct refractive errors of the eye. The intraocular lens may be made from a variety of materials or combination of materials such as PMMA, silicone, hydrogel and silicone hydrogel, etc.

In order to implant the intraocular lens into the eye, the sclera of the eye is incised, and a viscoelastic material is injected into the sclera to protect the anterior tissues. Then, the upper part of the lens capsule is incised to remove the clouded lens. Subsequently, the clouded lens nucleus is completely removed from the lens capsule, and the intraocular lens is injected into the lens capsule while injecting the viscoelastic material into the lens capsule to facilitate the insertion of the intraocular lens. The intraocular lens is injected into the lens capsule in a folded or rolled state and then restored to its original state, during which an intraocular lens injection device is used.

However, the conventional injection devices have the problems that the intraocular lens should be loaded in the device for the subsequent use, which makes it difficult to use, and the intraocular lens may be damaged due to deformation when it is loaded in the injection device using a pair of tweezers.

Moreover, when the intraocular lens is not accurately loaded in the injection device, the intraocular lens may be rotated, turned over, or damaged, and thus a person, not an expert cannot load the intraocular lens on the injection device and thus should be fully aware of the injection method for each injection device.

Furthermore, during the process of loading the intraocular lens on the injection device, the intraocular lens and the injection device may be contaminated due to improper sterilization.

### DISCLOSURE

### Technical Problem

Accordingly, the present invention has been made to solve the above-described problems, and an object of the present invention is to a disposable intraocular lens injection device, in which a receiving portion which is sealed after an intraocular lens is placed in an unfolded state therein, a cylinder including an injection guide disposed in front of the receiving portion and allowing the unfolded intraocular lens to be provided to a patient in a rolled state, and a plunger slidably disposed in the cylinder and injecting the intraocular lens in the receiving portion through the injection guide are packaged together with the injector device such that, since the intraocular lens is preloaded in the injection device, the difficulty for an operator to load the intraocular lens in the injection device during operation is overcome, thus making rapid progress while preventing bacteria infection during the operation.

### Technical Solution

To accomplish the above objects of the present invention, there is provided a disposable intraocular lens injection device in which an intraocular lens is preloaded, the device including: a cylinder in which a typical intraocular lens is loaded in an unfolded state; and a plunger penetrating the inside of the cylinder to inject the intraocular lens to a patient.

The cylinder may include a slide hole penetrating from one end to the other end of the cylinder.

A receiving portion may include a receiving base in which the intraocular lens is loaded in an unfolded state and a receiving cover covering the receiving base, and an injection guide may be provided on the other end of the receiving portion and include a first injection needle allowing the intraocular lens in the receiving portion to be provided to the patient in a rolled state and a second injection needle connected to the first injection needle, the receiving portion and the injection guide being provided at the other end of the cylinder.

The receiving base may extend from the bottom of the other end of the cylinder to the outside of the other end of the cylinder, a lower receiving groove may be formed on an upper surface of the receiving base in the longitudinal direction of the receiving base and connected to the slide hole, a pair of guide rails on which the intraocular lens is placed in an unfolded state may be provided on the lower receiving groove and extend in the longitudinal direction of the receiving base, one side of the receiving cover may be rotatably connected to one side of the receiving base, and an upper receiving groove facing the lower receiving groove may be formed on the receiving cover in the longitudinal direction of the receiving cover and connected to the slide hole.

The receiving base may further include a lower connection bracket extending from the other end of the receiving base to the outside, and the receiving cover may further include an upper connection bracket extending from the other end of the receiving cover to the outside, the lower connection bracket including a connection groove and the upper connection bracket including a connection hook inserted and connected to the connection groove, such that when the upper connection bracket is connected to the lower connection bracket, the intraocular lens may be defined between the receiving base and the receiving cover.

The receiving base and the receiving cover may include a lower supply hole and an upper supply hole, respectively, through which a viscoelastic material is injected.

The first injection needle may include a first injection guide hole connected to the lower receiving groove and the upper receiving groove, which face each other, and the second injection needle may include a second injection guide hole connected to the first injection guide hole, the first injection guide hole being radially narrowed from the lower and upper receiving grooves to the second injection guide hole and the second injection guide hole being formed horizontally.

The plunger may include a push rod inserted into the slide hole from the outside of the one end of the cylinder and an injection rod integrally formed with the other end of the push rod and inserted between the upper and lower receiving grooves to allow the intraocular lens to be provided to the patient.

A locking groove, into which an edge of an optical portion of the intraocular lens is inserted, may be concavely formed on the other end of the injection rod to prevent the intraocular lens from being turned over during the injection of the intraocular lens, and a seating groove, on which the leg portion of the intraocular lens is placed, may be formed to be depressed on the injection rod adjacent to the locking groove.

### Advantageous Effects

As described above, according to the present invention, since an intraocular lens is preloaded in an unfolded state in the injection device, it is possible to eliminate the difficulty of loading the intraocular lens in the injection device and prevent bacteria infection and damage of the intraocular lens. Moreover, it is not necessary to be fully aware of the method of loading the intraocular lens in the injection device, and thus its use is simple and convenient.

Further, it is possible to prevent the intraocular lens from being rotated and turned over during the injection of the intraocular lens and to make rapid progress by means of the guide rails formed on the lower receiving groove in which the intraocular lens is loaded, the first injection guide hole radially narrowed in the first injection needle such that the intraocular lens is rolled up during the injection, and the locking groove into which the intraocular lens is inserted is formed on the other end of the plunger.

### DESCRIPTION OF DRAWINGS

FIG. 1 is an exploded perspective view showing a disposable intraocular lens injection device in which an intraocular lens is preloaded according to the present invention;

FIG. 2 is a perspective view showing that a receiving portion of a cylinder shown in FIG. 1 is opened; and

FIG. 3 is an enlarged perspective view showing a plunger shown in FIG. 1.

### MODE FOR INVENTION

Hereinafter, a disposable intraocular lens injection device in which an intraocular lens is preloaded in accordance with exemplary embodiments of the present invention will be described with reference to the attached drawings.

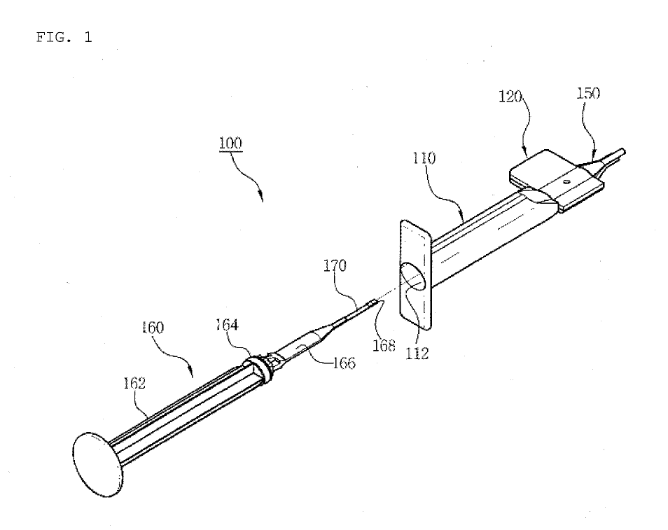

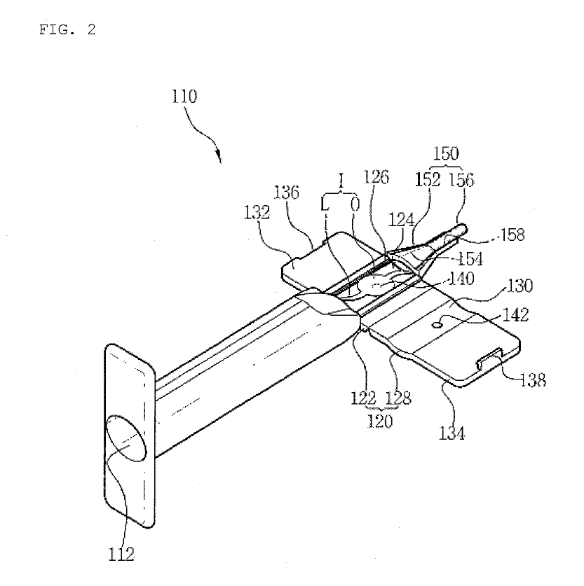

FIG. 1 is an exploded perspective view showing a disposable intraocular lens injection device in which an intraocular lens is preloaded in accordance with the present invention, and FIG. 2 is a perspective view showing that a receiving portion of a cylinder shown in FIG. 1 is opened.

In the following, the left side of the drawing will be referred to as “one end”, the right side of the drawing will be referred to as “the other end”, the upper side of the drawing will be referred to as the “top”, and the lower side of the drawing will be referred to as the “bottom”.

A disposable intraocular lens injection device 100 in which an intraocular lens is preloaded in accordance with the present invention includes a cylinder 110 and a plunger 160.

First, a slide hole 112 is provided in the cylinder 110. The slide hole 112 penetrates from one end to the other end of the cylinder 110. That is, the cylinder 110 has a hollow cylindrical shape with opened ends. Preferably, the cylinder 110 is made of a transparent material.

Moreover, a receiving portion 120 in which an intraocular lens I is placed in an unfolded state is provided on the other end of the cylinder 110, and an injection guide 150 is formed on the other end of the receiving portion 120 to allow the intraocular lens I in the receiving portion 120 to be provided to a patient in a rolled state.

The receiving portion 120 includes a receiving base 122 in which the intraocular lens I is placed and a receiving cover 128 covering the receiving base 122 in which the intraocular lens I has been placed.

The receiving base 122 extends from the bottom of the other end of the cylinder 110 to the outside of the other end of the cylinder 110 in the longitudinal direction of the cylinder 100. A lower receiving groove 124 is formed to be depressed on an upper surface of the receiving base 122. The lower receiving groove 124 is formed in the longitudinal direction of the receiving base 122 and connected to the slide hole 112. A pair of guide rails 126 on which the intraocular lens I is placed in an unfolded state is provided on the lower receiving groove 124. The guide rails 126 extend in the longitudinal direction of the receiving base 122. Preferably, an optical portion O of the intraocular lens I is placed in the middle of the guide rails 126, and a leg portion L of the intraocular lens I integrally formed with the optical portion O is placed in each of both ends the guide rails 126.

One side of the receiving cover 128 is rotatably connected to one side of the receiving base 122. An upper receiving groove 130 facing the lower receiving groove 124 is formed on the receiving cover 128. The upper receiving groove 130 is formed in the longitudinal direction of the receiving cover 128 and connected to the slide hole 112, like the lower receiving groove 124. Meanwhile, the receiving base 122 further includes a lower connection bracket 132 extending from the other end of the receiving base 122 to the outside, and the receiving cover 128 further includes an upper connection bracket 134 extending from the other end of the receiving cover 128 to the outside. The lower connection bracket 132 includes a connection groove 136, and the upper connection bracket 134 includes a connection hook 138 inserted and connected to the connection groove 136. That is, as the upper connection bracket 134 is connected to the lower connection bracket 132, the intraocular lens I is defined between the receiving base 122 and the receiving cover 128.

Preferably, the receiving base 122 and the receiving cover 128 include a lower supply hole 140 and an upper supply hole 142, respectively, through which a viscoelastic material is injected during the injection of the intraocular lens I.

Meanwhile, the injection guide 150 includes a first injection needle 152 integrally formed with the other end of the receiving base 122 and a second injection needle 156 integrally formed with the other end of the first injection needle 152.

The first injection needle 152 includes a first injection guide hole 154 connected to the lower receiving groove 124 and the upper receiving groove 130, which face each other, and the second injection needle 156 includes a second injection guide hole 158 connected to the first injection guide hole 154. The first injection guide hole 154 is radially narrowed from the lower and upper receiving grooves 124 and 130 to the second injection guide hole 158, and the second injection guide hole 158 is formed horizontally.

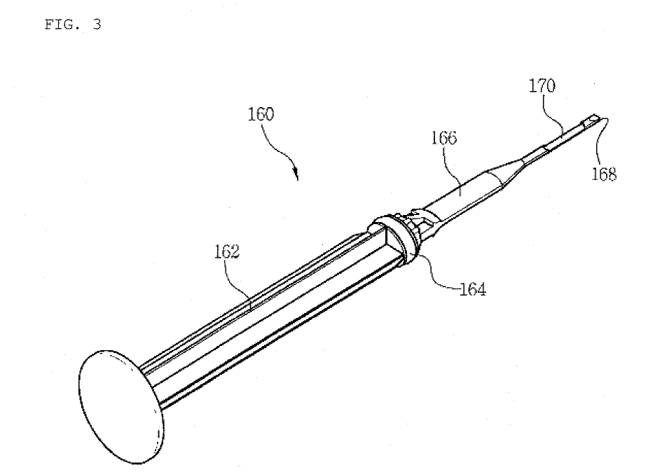

FIG. 3 is an enlarged perspective view showing a plunger shown in FIG. 1.

Referring to FIGS. 1 and 3, a plunger 160 includes a push rod 162 and an injection rod 166 integrally formed with the other end of the push rod 162. The push rod 162 and the injection rod 166 are inserted into the slide hole 112 from the outside of the one end of the cylinder 110. An O-ring 164, which is in sliding contact with the inner circumference of the slide hole 112, is mounted on the outer circumference of the push rod 162. Meanwhile, the injection rod 166 is slidably inserted into the lower and upper receiving grooves 124 and 130 and the first and second injection guide holes 154 and 158 to provide the intraocular lens I to a patient. A locking groove 168, into which an edge of the optical portion O of the intraocular lens I is inserted, is concavely formed on the other end of the injection rod 166, thereby preventing the intraocular lens I from being turned over during the injection of the intraocular lens I. Moreover, a seating groove 170, on which the leg portion L of the intraocular lens I is placed, is formed to be depressed on the injection rod 166 adjacent to the locking groove 168.

Next, the use of the disposable intraocular lens injection device 100, in which an intraocular lens I is preloaded, having the above-described configuration will be briefly described.

The injection device 100 according to the present invention is shipped from a factory in a state that the intraocular lens I is preloaded in the receiving portion 120 of the cylinder 110 and the plunger 160 is inserted into the cylinder 110. That is, in order to place the intraocular lens I on the receiving portion 120 in an unfolded state before the shipment, the intraocular lens I is first placed on the top of the guide rails 126 formed on the lower receiving groove 124 of the receiving base 122 such that the optical portion O of the intraocular lens I is placed on the top of the guide rails 126 and the leg portions L of the intraocular lens I are placed on both ends of the guide rails 126.

In this state, when the upper connection bracket 134 is rotated toward the lower connection bracket 132, the receiving cover 128 is put on the receiving base 122 and, when the connection hook 138 is connected to the connection groove 136, the intraocular lens I is defined between the lower receiving groove 124 and the upper receiving groove 130. When the intraocular lens I is loaded in the injection device 100 in the above-described manner, the plunger 160 is inserted into the one end of the cylinder 110, and the resulting injection device 100 is packed and shipped.

Meanwhile, in order to inject the intraocular lens I into the eye of a cataract patient using the packed injection device 100, the packed injection device 100 is unpacked, and a viscoelastic material is injected through the lower supply hole 140 and the upper supply hole 142. Then, in a state that the second injection needle 156 is placed adjacent to the patient's eye, the push rod 162 is pressed such that the other end of the injection rod 166 and the locking groove 168 formed on the other end of the injection rod 166 are inserted between the lower receiving groove 124 and the upper receiving groove 130 through the slide hole 112. As a result, the optical portion O of the intraocular lens I is inserted into the inside of the locking groove 168. Subsequently, when the push rod 162 is further pressed, the other end of the injection rod 166 guides the intraocular lens I toward the first injection guide hole 154. At this time, the intraocular lens I guided toward the first injection guide hole 154 is guided along the first injection guide hole 154 radially narrowed to be rolled up and then injected into the patient's eye through the second injection guide hole 158. Lastly, the injection device 100 is discarded upon completion of the injection.

### INDUSTRIAL APPLICABILITY

According to the disposable intraocular lens injection device of the present invention, in which an intraocular lens is preloaded in an unfolded state, it is possible to eliminate the difficulty of loading the intraocular lens in the injection device, and prevent bacteria infection and damage of the intraocular lens. Moreover, it is not necessary to be fully aware of the method of loading the intraocular lens in the injection device, and thus its use is simple and convenient.

Further, it is possible to prevent the intraocular lens from being rotated and turned over during the injection of the intraocular lens and to make rapid progress with the use of the injection device in accordance with the present invention.

It will be apparent to those skilled in the art that various modifications can be made to the above-described exemplary embodiments of the present invention without departing from the spirit or scope of the invention. Thus, it is intended that the present invention covers all such modifications provided they come within the scope of the appended claims and their equivalents.

1. A disposable intraocular lens injection device in which an intraocular lens is preloaded, the injection device comprising:
a cylinder in which a typical intraocular lens is loaded in an unfolded state; and
a plunger penetrating the inside of the cylinder to inject the intraocular lens to a patient.

2. The injection device of claim 1, wherein the cylinder comprises a slide hole penetrating from one end to the other end of the cylinder.

3. The injection device of claim 2, wherein a receiving portion including a receiving base in which the intraocular lens is loaded in an unfolded state and a receiving cover covering the receiving base, and an injection guide provided on the other end of the receiving portion and including a first injection needle allowing the intraocular lens in the receiving portion to be provided to the patient in a rolled state and a second injection needle connected to the first injection needle are provided at the other end of the cylinder.

4. The injection device of claim 3, wherein the receiving base extends from the bottom of the other end of the cylinder to the outside of the other end of the cylinder, a lower receiving groove is formed on an upper surface of the receiving base in the longitudinal direction of the receiving base and connected to the slide hole, a pair of guide rails on which the intraocular lens is placed in an unfolded state is provided on the lower receiving groove and extends in the longitudinal direction of the receiving base, one side of the receiving cover is rotatably connected to one side of the receiving base, and an upper receiving groove facing the lower receiving groove is formed on the receiving cover in the longitudinal direction of the receiving cover and connected to the slide hole.

5. The injection device of claim 4, wherein the receiving base further comprises a lower connection bracket extending from the other end of the receiving base to the outside, and the receiving cover further comprises an upper connection bracket extending from the other end of the receiving cover to the outside, the lower connection bracket including a connection groove and the upper connection bracket including a connection hook inserted and connected to the connection groove, such that when the upper connection bracket is connected to the lower connection bracket, the intraocular lens is defined between the receiving base and the receiving cover.

6. The injection device of claim 4, wherein the receiving base and the receiving cover comprise a lower supply hole and an upper supply hole, respectively, through which a viscoelastic material is injected.

7. The injection device of claim 4, wherein the first injection needle comprises a first injection guide hole connected to the lower receiving groove and the upper receiving groove, which face each other, and the second injection needle comprises a second injection guide hole connected to the first injection guide hole, the first injection guide hole being radially narrowed from the lower and upper receiving grooves to the second injection guide hole and the second injection guide hole being formed horizontally.

8. The injection device of claim 4, wherein the plunger comprises a push rod inserted into the slide hole from the outside of the one end of the cylinder and an injection rod integrally formed with the other end of the push rod and inserted between the upper and lower receiving grooves to allow the intraocular lens to be provided to the patient, and wherein an O-ring which is in sliding contact with the inner circumference of the slide hole is mounted on the outer circumference of the push rod.

9. The injection device of claim 8, wherein a locking groove, into which an edge of an optical portion of the intraocular lens is inserted, is concavely formed on the other end of the injection rod to prevent the intraocular lens from being turned over during the injection of the intraocular lens, and a seating groove, on which the leg portion of the intraocular lens is placed, is formed to be depressed on the injection rod adjacent to the locking groove.
